(19) United States
(12) Patent Application Publication
(10) Pub. No.: US 2026/0049218 A1
(43) Pub. Date: Feb. 19, 2026

(54) INTERFERENCE PIGMENTS

(71) Applicant: MERCK PATENT GmbH, Darmstadt (DE)

(72) Inventors: Carsten Handrosch, Darmstadt (DE); Martin Knapp, Darmstadt (DE); Carsten Plueg, Darmstadt (DE); Sabine Schoen, Darmstadt (DE); Lars Dobelmann-Mara, Darmstadt (DE)

(73) Assignee: MERCK PATENT GmbH, Darmstadt (DE)

(21) Appl. No.: 19/279,990

(22) Filed: Jul. 24, 2025

(30) Foreign Application Priority Data
Aug. 16, 2024 (EP) 24195014

(51) Int. Cl. C09C 1/00; A23L 5/42; A61K 8/02; A61Q 1/02; C09C 3/10; C09D 11/037; C09D 11/50

(52) U.S. Cl. CPC C09C 1/0021; A23L 5/42; A61K 8/0258; A61Q 1/02; C09C 3/10; C09D 11/037; C09D 11/50; A61K 2800/436; C01P 2004/20; C01P 2004/62; C01P 2004/84; C01P 2006/60; C09C 2200/1004; C09C 2200/301; C09C 2200/308; C09C 2200/408; C09C 2220/106

(57) ABSTRACT

Interference pigments based on SiO₂ flakes coated with crystallites of alpha-Fe₂O₃. Also, a process for the preparation of interference pigments that comprise SiO₂ flakes coated with a crystallite of alpha-Fe₂O₃, by a wet-chemical process in a fluidised bed, by a CVD process, by a PVD process, or by any combination of said processes. Also, the use of interference pigments based on SiO₂ flakes coated with crystallites of alpha-Fe₂O₃, in particular in paints, coatings, industrial and automobile paints, ceramic materials, plastics and cosmetic formulations.

### CROSS-REFERENCE TO RELATED APPLICATIONS

This is a U.S. nonprovisional patent application filed under 35 U.S.C. § 111 (a), claiming priority benefit under 35 U.S.C. § 119 (a) of and to European Patent Application No. 24195014.6, filed Aug. 16, 2024, the contents of which document are incorporated herein by reference in their entirety and for all purposes.

### FIELD OF INVENTION

The present invention relates to interference pigments based on SiO₂ flakes coated with alpha-Fe₂O₃ crystallites, and to the use of the pigments, in particular in paints, coatings, printing inks, plastics and cosmetic formulations.

### BACKGROUND

WO 93/08237 discloses interference pigments based on transparent silicon dioxide flakes. The pigments described therein are based on SiO₂ flakes coated with one or more metal oxide layers. Instead of metal oxides, the SiO₂ flakes can also be coated with other materials, such as, for example, metals, sulfides or nitrides. Intensely coloured red interference pigments based on SiO₂ flakes coated with iron oxide are, for example, the subject-matter of WO 2007/057111 A2 and EP 1 681 318 A2.

The red interference pigments known from the prior art exhibit a strong orange-red colour effect at a steep viewing angle, which is a combination of interference colour and absorption colour, and very high hiding power.

### SUMMARY OF THE INVENTION

The object of the present invention is to provide a highly opaque, mass-tone-coloured interference pigment having a combination of bright bluish-red absorption colour and high-chromaticity bluish-red interference colour, in which a low colour flop occurs and the bluish-red absorption colour accordingly remains visible even at a flat viewing angle, so that the intensity of the interference colour, which decreases naturally at a flat viewing angle, is compensated by the absorption colour, which is bright at all viewing angles. As a result, no orange or even dark-brown down-flop is observed. In particular, the aim is to provide an interference pigment which has a bright red hue at all viewing angles. Furthermore, the pigment should have a high hiding power at the same time as low texture and be usable for vegan applications.

Surprisingly, it has been found that interference pigments based on SiO₂ flakes and coated with alpha-Fe₂O₃ crystallites of defined size have none of the above-mentioned disadvantages and exhibit a pure and bright bluish-red hue at all viewing angles. The pigments are distinguished by their relatively high hiding power and high colour saturation, are temperature-stable and can be employed universally in a very wide variety of applications.

The interference pigments according to the invention exhibit an opaque, pure, bluish-red absorption colour on all substrates (white/black) and in addition have only a low angle dependence of the superimposed interference colour. The overall colour effect according to the invention (absorptive colour+interference colour) that arises is therefore a pure bluish-red hue at all viewing angles (steep/flat). From an applicational point of view, the interference pigments according to the invention having the combination of:
chromatic, blue-red mass tone
intense bluish-red interference colour
low angle dependence of the colour impression/low colour flop
high hiding power are particularly suitable for the colouring of coating formulations, paints and plastics.

Furthermore, the very good feel on the skin predestines use in cosmetic formulations. Very particularly advantageous in this area of application is the possibility of producing vegan cosmetics in blends with white pigments, since these pigment mixtures can replace the classical Carmine Red in pink formulations.

The present invention relates to interference pigments based on SiO₂ flakes, characterised in that they are coated with crystallites of alpha-haematite (alpha-Fe₂O₃).

Owing to the advantageous properties, the interference pigments according to the invention are universally suitable for a multiplicity of applications of a wide variety of types. Accordingly, the present invention also relates to the use of these pigments in paints, coatings, industrial and automobile paints, printing inks, security printing inks, paper, plastics, films, cosmetic formulations, button pastes, pigment mixtures, dry preparations or pigment preparations, ceramic materials, ceramic colours, glazes, engobes, enamels and glasses, as absorbers for the laser marking of plastics, for the colouring of foods, for the finishing of foods, in food and medicament coatings, for security features in documents and identity papers, for the colouring of seed, for RADAR applications and for the colouring of solar cells.

### DETAILED DESCRIPTION OF THE INVENTION

The pigments according to the invention are based on synthetic SiO₂ flakes as substrate, which generally have a uniform layer thickness and are preferably produced in accordance with international application WO 93/08237 by solidification and hydrolysis of a water-glass solution on a continuous belt. Uniform layer thickness here is taken to mean a layer thickness tolerance of 3 to 10%, preferably 3 to 5%, of the total dry-layer thickness of the particles. The flake-form silicon dioxide particles are generally in amorphous form. Synthetic flakes of this type have the advantage over natural materials, such as, for example, mica, that the layer thickness can be adjusted in view of the desired effects and the layer thickness tolerance is limited.

Suitable SiO₂ flakes for the interference pigments according to the invention preferably have a diameter of 1 to 250 μm, in particular 1-65 μm. The thickness of the SiO₂ flakes is preferably 250-500 nm, in particular 330 to 400 nm, and the average layer thickness of the α-Fe₂O₃ layer in the pigment is 85+/−35 nm.

Essential for the interference pigments according to the invention are the crystallites on the surface of the SiO₂ flakes. The crystallites are distributed unevenly on the surface of the SiO₂ flakes and consist of α-Fe₂O₃. The average size of the crystallites is preferably ≥45 nm, in particular ≥50 nm.

The nucleation and the crystallite size can be influenced or adjusted by, inter alia, the reaction temperature and the stirring speed as well as the duration of the metered addition and by the calcination temperature and residence time in the calcination oven.

Besides the crystallites and their size, the total thickness of the interference pigment is essential for the properties of the pigment. The thickness of the SiO₂ flakes and the crystallite layer is preferably selected so that the total thickness of the interference pigments, on unweighted arithmetic average, is not greater than 494 nm (+/−109 nm).

The coating of the SiO₂ flakes on the surface with the crystallites of α-Fe₂O₃ can be carried out by wet-chemical methods and/or by CVD or PVD processes. The interference pigments according to the invention are preferably prepared by wet-chemical methods, in which the known wet-chemical coating technologies developed for the preparation of pearlescent pigments can be applied.

For coating, the SiO₂ flakes are suspended in water and coated by slow and controlled addition and precipitation of corresponding inorganic iron compounds, during which the pH necessary for the precipitation of α-Fe₂O₃ and the nucleation is set and kept constant by simultaneous addition of acid or base, and the substrate coated with the crystallites is subsequently separated off from the aqueous suspension, dried and calcined.

The calcination temperature is 700 to 1000° C., preferably 800 to 950° C., in order to ensure that the crystallites contain no FeOOH, but consist exclusively of α-Fe₂O₃. The high calcination temperature also results in greater densification of the crystallites, which in turn leads to a higher refractive index. The latter is responsible for, inter alia, the high luminance of the interference pigments according to the invention. The duration of the calcination process is generally 30 to 60 minutes. A longer residence time in the calcination oven is advantageous for the desired densification of the crystallites.

CVD or PVD processes for coating the SiO₂ flakes with crystallites of α-Fe₂O₃ are also suitable for the preparation of the interference pigments according to the invention.

In order to increase the light, water and weather stability, it is frequently advisable, depending on the field of application, to subject the interference pigment according to the invention to inorganic or organic post-coating or post-treatment. Suitable post-coatings or post-treatments are, for example, the processes described in EP 0 632 109, U.S. Pat. No. 5,759,255, DE 43 17 019, DE 39 29 423, DE 32 35 017, EP 0 492 223, EP 0 342 533, EP 0 268 918, EP 0 141 174, EP 0 764 191, WO 98/13426 or EP 0 465 805, the disclosure content of which is hereby incorporated herein by way of reference. This post-coating further increases the chemical and photochemical stability or simplifies handling of the interference pigment, in particular incorporation into various media. In order to improve the wettability, dispersibility and/or compatibility with the user media, functional coatings comprising SiO₂, SnO₂, Al₂O₃ or ZrO₂ or mixtures thereof can be applied to the pigment surface. Furthermore, organic post-coatings are possible, for example with silanes, as described, for example, in EP 0090259, EP 0 634 459, WO 99/57204, WO 96/32446, WO 99/57204, U.S. Pat. Nos. 5,759,255, 5,571,851, WO 01/92425 or in J. J. Ponjeé, Philips Technical Review, Vol. 44, No. 3, 81 ff. and P. H. Harding J. C. Berg, J. Adhesion Sci. Technol. Vol. 11 No. 4, pp. 471-493. Interference pigments containing an organic coating, for example comprising organosilanes or organotitanates or organozirconates, exhibit not only the above-mentioned optical properties, but additionally increased stability to weathering influences, such as, for example, moisture and light, which is of particular interest for industrial coatings and in the automobile sector. The stabilisation can be improved by inorganic components of the additional coating. Overall, the respective proportions for the additional stabilising coating should be selected so that the optical properties of the interference pigments according to the invention are not significantly affected. The substances applied here merely encompass a proportion by weight of 0.1 to 5% by weight, preferably 0.5 to 3% by weight, of the total pigment.

The post-coating of the interference pigments according to the invention can be carried out directly on the α-Fe₂O₃ crystallite coating of the SiO₂ flakes in a one-pot process. However, it is also possible firstly to isolate the interference pigment, dry it and calcine it and then apply the post-coating.

In this patent application, coating(s) is taken to mean the complete covering/enveloping of the flake-form substrates.

The hiding power of the interference pigments according to the invention can furthermore be increased if the pigments are mixed in combination with organic and inorganic fillers and/or with flake-form, needle-shaped, spherical or crystalline colourants. The admixing of one or more colourants with the interference pigments according to the invention enables colour effects to be enhanced and novel colour effects to be achieved. The present invention therefore likewise relates to pigment mixtures.

Owing to the measurably larger average crystallite size, the interference pigments according to the invention exhibit increased temperature and heat stability compared with the iron oxide pigments known from the prior art and can therefore easily be incorporated into engobes and glazes. Depending on the desired effect, the glazes can be matt to glossy, or transparent to opaque. The invention furthermore also relates to formulations, such as, for example, ceramic colours, coatings, ceramic tiles, cast ceramics, sanitary ceramics, enamels, glazes, clay-, glass- and ceramicware, which comprise the interference pigment according to the invention.

The interference pigments according to the invention are furthermore suitable for the preparation of flowable pigment preparations and dry preparations, in particular for printing inks and paints, preferably automotive paints, consisting of the pigments according to the invention, binders and optionally one or more additives.

The interference pigment according to the invention is compatible with a multiplicity of colour systems, preferably from the area of paints, coatings and printing inks. A multiplicity of binders, particularly water-soluble types, as marketed, for example, by BASF, Marabu, Pröll, Sericol, Hartmann, Gebr. Schmidt, Sicpa, Aarberg, Siegwerk, GSBWahl, Follmann, Ruco or Coates Screen INKS GmbH, are suitable for the preparation of printing inks for, for example, gravure printing, flexographic printing, offset printing and offset overprint varnishing. The printing inks can be water-based or solvent-based.

It goes without saying that for the various applications, the interference pigment according to the invention can also advantageously be employed as a blend with, for example,
metal-effect pigments, for example based on iron flakes or aluminium flakes;
pearlescent pigments based on metal oxide-coated synthetic mica flakes, natural mica flakes, glass flakes, Al₂O₃ flakes, Fe₂O₃ flakes or SiO₂ flakes;
absorption pigments;
goniochromatic pigments;
multilayered pigments (preferably comprising 2, 3, 4, 5 or 7 layers) based on metal oxide-coated synthetic mica flakes, natural mica flakes, glass flakes, Al₂O₃ flakes, Fe₂O₃ flakes or SiO₂ flakes;
organic dyes;
organic pigments;
inorganic pigments, such as, for example, transparent and opaque white, coloured and black pigments; in particular, temperature-stable ceramic pigments;
flake-form iron oxides;
carbon black;
ceramic colour bodies;
functional pigments, for example IR-reflective or electrically conductive pigments.

In a preferred embodiment, the interference pigment according to the invention is advantageously mixed with organic and inorganic coloured pigments and dyes of natural or synthetic origin, such as, for example, Carmine Red, chromium oxide, ultramarine or spherical SiO₂ or TiO₂ pigments. The mixing ratio depends on the application medium and the effect to be achieved.

The interference pigment according to the invention can be mixed in any ratio with commercially available pigments and/or other commercially available fillers. For use as a Carmine Red substitute in vegan cosmetic formulations, explicit mention may be made here of blending with silver-white interference pigments, titanium dioxide or other white pigments. The various mixing ratios allow coverage of the desired colour nuances in a wide range of pinks.

Commercially available fillers that may be mentioned are, for example, natural and synthetic mica, glass beads, glass powder, nylon powder, pure or filled melamine resins, talc, glasses, kaolin, oxides or hydroxides of aluminium, magnesium, calcium, zinc, BiOCl, barium sulfate, calcium sulfate, calcium carbonate, magnesium carbonate, carbon, boron nitride and physical or chemical combinations of these substances. There are no restrictions regarding the particle shape of the filler. Depending on requirements, it can be irregularly shaped, flake-form, spherical or needle-shaped, crystalline or amorphous.

The interference pigment according to the invention is particularly suitable for decorative cosmetics and for personal care applications, such as, for example, nail varnishes, lipsticks, lip gloss, rouge, compact powders, gels, lotions, soaps, toothpaste, body lotions, emulsions, soaps, shampoos, BB creams, CC creams, make-up, foundations, (volume) mascara, hair, eyelash and eyebrow products, sun protection, pre-sun and after-sun preparations, make-ups, body lotions, bath gels, soaps, bath salts, toothpaste, hair gels, compact powders, loose powders, etc.

In decorative cosmetics, the interference pigment according to the invention is preferably employed in concentrations of 0.5-25% by weight, in particular 1-20% by weight and very particularly preferably 1-10% by weight, based on the formulation. In cosmetic formulations for personal care applications, the interference pigment according to the invention is preferably employed in concentrations of 0.1-5% by weight and very particularly preferably 0.5-4% by weight, based on the formulation.

The interference pigments according to the invention can of course also be combined with cosmetic raw materials and assistants of any type. These include, inter alia, oils, fats, waxes, film formers, surfactants, antioxidants, such as, for example, vitamin C or vitamin E, stabilisers, odour enhancers, silicone oils, emulsifiers, solvents, such as, for example, ethanol, ethyl acetate or butyl acetate, preservatives and assistants which generally determine the technical applicational properties, such as, for example, thickeners and rheological additives, such as, for example, bentonites, hectorites, silicon dioxides, Ca silicates, gelatines, high-molecular-weight carbohydrates and/or surface-active assistants, etc.

The formulation comprising the interference pigment according to the invention can belong to the lipophilic, hydrophilic or hydrophobic type. In heterogeneous formulations with discrete aqueous and non-aqueous phases, the interference pigment according to the invention may be present in only one of the two phases or distributed across both phases.

The pH values of the formulations can be between 1 and 14, preferably between 2 and 11 and particularly preferably between 4 and 10.

There are no limits to the concentrations of the interference pigment according to the invention in the formulation. Depending on the application, they can be between 0.001 (rinse-off products, for example shower gels) and 60%. The interference pigment according to the invention can furthermore also be combined with cosmetic active compounds. Suitable active compounds are, for example, insect repellents, inorganic UV filters, such as, for example, TiO₂, UV A/BC protection filters (for example OMC, B3, MBC), including in encapsulated form, anti-aging active compounds, vitamins and derivatives thereof (for example vitamins A, C, E, etc.), self-tanning agents (for example DHA, erythrulose, etc.) and other cosmetic active compounds, such as, for example, bisabolol, LPO, ectoine, emblica, allantoin, bioflavanoids and derivatives thereof.

Organic UV filters are generally incorporated into cosmetic formulations in an amount of 0.5 to 10 percent by weight, preferably 1-8%, and inorganic filters in an amount of 0.1 to 30%.

In addition, the formulations may comprise conventional skin-protecting or skin-care active compounds, such as, for example, aloe vera, avocado oil, coenzyme Q10, green tea extract and active-compound complexes. Particularly preferred active compounds are pyrimidinecarboxylic acids and/or aryl oximes.

Of the cosmetic applications, particular mention should be made of the use of ectoine and ectoine derivatives for the care of aged, dry or irritated skin. European patent application EP-A-0 671 161 describes, in particular, that ectoine and hydroxyectoine are employed in cosmetic preparations, such as powders, soaps, surfactant-containing cleansing products, lipsticks, rouge, make-up, skin care creams and sun-protection preparations.

Application forms of cosmetic formulations that may be mentioned are, for example: solutions, suspensions, emulsions, PIT emulsions, pastes, ointments, gels, creams, lotions, powders, soaps, surfactant-containing cleansing preparations, oils, aerosols and sprays. Other application forms are, for example, sticks, shampoos and shower gels. Any desired usual vehicles, assistants and optionally further active compounds may be added to the preparation.

Ointments, pastes, creams and gels may comprise the usual vehicles, for example animal and vegetable fats, waxes, paraffins, starch, tragacanth, cellulose derivatives, polyethylene glycols, silicones, bentonites, silicic acid, talc and zinc oxide, or mixtures of these substances.

Powders and sprays may comprise the usual vehicles, for example lactose, talc, silicic acid, aluminium hydroxide, calcium silicate and polyamide powder, or mixtures of these substances. Sprays may additionally comprise the usual propellants, for example chlorofluorocarbons, propane/butane or dimethyl ether.

Solutions and emulsions may comprise the usual vehicles, such as solvents, solubilisers and emulsifiers, for example water, ethanol, isopropanol, ethyl carbonate, ethyl acetate, benzyl alcohol, benzyl benzoate, propylene glycol, 1,3-butyl glycol, oils, in particular cottonseed oil, peanut oil, corn germ oil, olive oil, castor oil and sesame oil, glycerol fatty acid esters, polyethylene glycols and fatty acid esters of sorbitan, or mixtures of these substances.

Suspensions may comprise the usual vehicles, such as liquid diluents, for example water, ethanol or propylene glycol, suspending agents, for example ethoxylated isostearyl alcohols, polyoxyethylene sorbitol esters and polyoxyethylene sorbitan esters, microcrystalline cellulose, aluminium metahydroxide, bentonite, agar-agar and tragacanth, or mixtures of these substances.

Soaps may comprise the usual vehicles, such as alkali-metal salts of fatty acids, salts of fatty acid semi-esters, fatty acid protein hydrolysates, isothionates, lanolin, fatty alcohol, vegetable oils, plant extracts, glycerine, sugar or mixtures of these substances.

Surfactant-containing cleansing products may comprise the usual vehicles, such as salts of fatty alcohol sulfates, fatty alcohol ether sulfates, sulfosuccinic acid semiesters, fatty acid protein hydrolysates, isothionates, imidazolinium derivatives, methyl taurates, sarcosinates, fatty acid amide ether sulfates, alkylamidobetaines, fatty alcohols, fatty acid glycerides, fatty acid diethanolamides, vegetable and synthetic oils, lanolin derivatives, ethoxylated glycerol fatty acid esters, or mixtures of these substances.

Face and body oils may comprise the usual vehicles, such as synthetic oils, such as, for example, fatty acid esters, fatty alcohols, silicone oils, natural oils, such as vegetable oils and oily plant extracts, paraffin oils, lanolin oils or mixtures of these substances.

The cosmetic preparations can be in various forms. Thus, they can be, for example, a solution, an anhydrous preparation, an emulsion or microemulsion of the water-in-oil (W/O) type or of the oil-in-water (O/W) type, a multiple emulsion, for example of the water-in-oil-in-water (W/O/W) type, a gel, a solid stick, an ointment or also an aerosol. It is also advantageous to administer ectoines in encapsulated form, for example, in collagen matrices and other usual encapsulation materials, for example as cellulose encapsulations, in gelatine, wax matrices or liposomally encapsulated. In particular, wax matrices, as described in DE-A 43 08 282, have proven favourable. Emulsions are preferred. O/W emulsions are particularly preferred. Emulsions, W/O emulsions and O/W emulsions can be obtained in the usual way.

Further embodiments are oily lotions based on natural or synthetic oils and waxes, lanolin, fatty acid esters, in particular triglycerides of fatty acids or oily-alcoholic lotions based on a lower alcohol, such as ethanol, or a glycerol, such as propylene glycol, and/or a polyol, such as glycerine, and oils, waxes and fatty acid esters, such as triglycerides of fatty acids.

Solid sticks consist of natural or synthetic waxes and oils, fatty alcohols, fatty acids, fatty acid esters, lanolin and other fatty bodies.

If a preparation is formulated as an aerosol, the usual propellants are generally used, such as alkanes, fluoroalkanes and chlorofluoroalkanes.

The cosmetic preparation can also be used for the protection of hair against photo-chemical damage, for the prevention of colour nuance changes, bleaching or mechanical damage. In this case, formulation as a shampoo, lotion, gel or emulsion for rinsing off is suitable, with the respective preparation being applied before or after shampooing, before or after colouring or bleaching or before or after a permanent set. A preparation can also be selected as a lotion or gel for styling and treatment, as a lotion or gel for brushing or setting a water wave, as a hair lacquer, perm, hair colouring or hair bleaching product. The preparation with light-protective properties may comprise adjuvants, such as interface-active agents, thickeners, polymers, softening agents, preservatives, foam stabilisers, electrolytes, organic solvents, silicone derivatives, oils, waxes, anti-grease agents, dyes and/or pigments which colour the composition itself or the hair, or other ingredients usually used for hair care.

The present invention likewise relates to formulations, in particular formulations which, besides the interference pigment according to the invention, comprise at least one constituent selected from the group of the absorbents, astringents, antimicrobial substances, antioxidants, antiperspirants, antifoams, antidandruff active compounds, antistatics, binders, biological additives, bleaching agents, chelating agents, deodorants, emollients, emulsifiers, emulsion stabilisers, dyes, humectants, film formers, fillers, fragrances, flavourings, insect repellents, preservatives, corrosion inhibitors, cosmetic oils, solvents, oxidants, plant constituents, buffer substances, reducing agents, surfactants, propellant gases, opacifiers, UV filters and UV absorbers, denaturing agents, aloe vera, avocado oil, coenzyme Q10, green tea extract, viscosity regulators, perfume, inorganic pigments, such as, for example, transparent or opaque white, coloured and black pigments, metal pigments, temperature-stable ceramic pigments, ceramic colour bodies, functional pigments, such as, for example, IR-reflecting pigments or electrically conductive pigments, and vitamins.

The interference pigment according to the invention can be employed for the pigmentation of food colourings, for the finishing of foods, in food coatings, for example mass colouring or as a coloured coating, in medicament coatings, for example in sugarcoated pills and tablets.

The colouring of pharmaceutical and food products is carried out by adding the interference pigment, in the desired mixing ratios, to the product to be coloured in amounts of 0.005 to 15% by weight, preferably 0.01 to 100% by weight.

The admixture of food-approved natural or nature-identical dyes, organic or inorganic coloured pigments or colouring natural fruit and plant extracts enables the colour effect of the interference pigments according to the invention in the product to be influenced and at the same time novel iridescent colour effects to be achieved.

Suitable natural or nature-identical dyes are, in particular, E 101, E 104, E 110, E 124, E 131, E 132, E 140, E 141, E 151 and E 160a. Furthermore, other coloured pigments can also be admixed with the interference pigments according to the invention, such as, for example, E 171, E 172 and E 153.

The proportion of dyes besides the interference pigments according to the invention, based on the food or pharmaceutical product, is preferably in the range from 0.5 to 25% by weight. Fruit and plant extracts, such as, for example, carrot juice, beetroot juice, elderberry juice, hibiscus juice, paprika extract and chokeberry extract, can likewise be employed as dye.

The total concentration of all pigments in the product to be pigmented should not exceed 50% by weight, based on the product. It is generally dependent on the specific application.

Various active compound admixtures, such as, for example, vitamins, enzymes, trace elements, proteins, carbohydrates, essential fats and/or minerals, may also be added to the food and pharmaceutical products, where the total amount of active compounds, based on the food or pharmaceutical product, should not exceed 25% by weight. The amount of active compounds or active compound mixtures is preferably 0.01-20% by weight, based on the product.

The colouring of the products is carried out by adding the interference pigment to the product to be coloured, alone or in combination with further pigments or colourants, directly or in the presence of water and/or an organic solvent, in the desired mixing ratios, simultaneously or successively, during or after preparation thereof, before or after shaping (for example, in the case of extrusion, pelleting, expansion, granulation, etc.). It is likewise possible to admix the interference pigments according to the inven-tion with pulverulent or loose powders.

For the colouring of food and pharmaceutical products, the interference pigments according to the invention, alone or in a pigment mixture, can also be applied to the surface after shaping. In this case, the interference pigment is generally mixed with an application medium and subsequently applied to the product using suitable application and spray devices. The application or coating composition then ensures corresponding adhesion of the interference pigments to the product surface. The latter is then coloured correspondingly.

In the case of incorporation into the product matrix itself, the amount of the interference pigments according to the invention used is preferably 0.5-40% by weight, in particular 1-30% by weight. In the case of the surface colouring of food and pharmaceutical products, the use range in the colouring or coating solution used is 0.1-25% by weight, in particular 1-15% by weight. In the case of the use of the interference pigments according to the invention in pulverulent products, the use range is 0.05-50% by weight, in particular 2-10% by weight.

The coating solutions preferably comprise water or organic solvents, such as, for example, ethanol or isopropanol. The film former employed in the coating solutions is preferably a cellulose derivative, such as, for example, hydroxypropylmethylcellulose. Particularly preference is given to application solutions comprising cellulose derivatives that comprise 5-80% by weight of a suitable organic solvent instead of water.

Alcoholic or alcoholic/aqueous cellulose-containing application solutions have significant applicational advantages over aqueous coating solutions:
use of cooler drying air during spray application
colouring of heat-sensitive products, such as, for example, vitamin-containing foods, with the red interference pigments is very readily possible.

Products suitable for colouring that may be mentioned in particular are coatings on foods of all types, in particular pigmented sugar and shellac coatings (alcoholic and aqueous), coatings with oils and waxes, with gum arabic and with cellulose types (for example HPMC=hydroxypropylmethylcellulose), with starch and protein derivatives, carrageen and other substances suitable for coating that are known to the person skilled in the art. In this case, the interference pigment according to the invention is generally mixed with the application medium and subsequently applied to the food or pharmaceutical product using suitable application and spray devices or by hand. The application or coating composition then ensures corresponding adhesion of the pigments to the surface of the food or pharmaceutical product. This surface is then coloured correspondingly. The application and coating solutions preferably comprise 0.1-20% by weight, in particular 2-15% by weight, of interference pigments.

Preferred dry-powder mixtures for coatings comprise a cellulose derivative, such as, for example, hydroxypropylmethylcellulose, sodium carboxymethylcellulose, a release agent, such as, for example, lecithin or stearic acid, a gloss enhancer, such as, for example, maltodextrin and/or dextrose, and the interference pigment according to the invention. Dry-powder mixtures of this type preferably comprise the interference pigment according to the invention in amounts of 0.01-50% by weight, in particular 0.5-40% by weight, based on the powder mixture. Dyes, flavourings, vitamins, sweeteners, etc., can also be added to these dry-powder mixtures as required.

Products suitable for colouring or coating are, for example, confectionery, cake decorations, tablets, sugar-coated pills, chewing gums, gum products, fondant products, marzipan products, filling compositions, cocoa and fat glazes, chocolate and chocolate-containing products, ice cream, cereals, snack products, coatings, cake coating compositions, sugar sprinkles, nonpareils, jelly and gelatine products, candies, liquorice, sugar icing, candyfloss, fat, sugar and cream compositions, puddings, desserts, cake glazes, cold dishes, lemonades and fizzy drinks, drinks with stabilising additives, such as, for example, carboxymethylcellulose, soured and unsoured milk products, such as, for example, quark, yogurt, cheese, cheese rinds, sausage casings, etc.

In the case of sugar-coated or coated food and pharmaceutical products, combination of the interference pigments according to the invention with flavourings (powder or liquid flavourings), acids and/or sweeteners, such as, for example, aspartame, is possible in order to reinforce the visual effect in taste terms too.

The invention therefore relates to all formulations from the food and pharmaceutical sectors that comprise the interference pigments according to the invention alone or in combination with further pigments/pigment mixtures or dyes (natural or nature-identical) as colourant.

A further large area of application is in the pharmaceutical and OTC sector for colouring or as a coating for tablets, gelatine capsules, sugar-coated pills, ointments, cough syrup, etc. In combination with conventional coatings, such as polymethacrylates and cellulose types, for example HPMC, the interference pigment according to the invention can be employed in a variety of ways for colouring and finishing the products.

The invention furthermore relates to the use of the interference pigments according to the invention in paints, coatings, printing inks, security printing inks, plastics, ceramic materials, glasses, glazes, as tracers, as absorbers for the laser marking of plastics and papers, in cosmetic formulations, for the pigmentation of food colourings, for the finishing of foods, for example mass colouring or as a coloured coating, and in medicament coatings, for example in sugar-coated pills and tablets. Furthermore, the pigments according to the invention are also suitable for the preparation of pigment preparations and for the preparation of dry preparations, such as, for example, granules, chips, pellets, briquettes, etc. The dry preparations are particularly suitable for paints and printing inks.

Since the pigment according to the invention is RADAR-transparent, it is also suitable for RADAR applications, especially in the automobile sector.

Furthermore, the interference pigment according to the invention can be used for the colouring of solar cells, for example by the application of a paint comprising the pigment according to the invention to the solar panel.

The examples below are intended to illustrate the invention, but without limiting it. Unless otherwise stated, percent figures are by weight.

### EXAMPLES

### Example 1

100 g of SiO₂ flakes (average flake thickness 365 nm) are suspended in 2 l of demineralised water and heated to 85° C. with stirring. Sufficient FeCl₃ solution (content 7% of Fe) is then metered in until the desired cut-off point has been reached. During the metered addition, the pH of the suspension is kept constant at 3.1 by addition of 30% KOH.

When the addition of FeCl₃ is complete, the pH of the suspension is adjusted to 6.5 using 30% KOH. The pigment precursor is subsequently filtered off, washed and dried at 110° C. Finally, the pigment is calcined at 900° C. and sieved.

The bluish-red interference pigment obtained exhibits the following L,a,b values:

Paint card over black (measured using a Byk-mac I spectrophotometer, measurement angle) 75°:

$${L}^{*} = 14.07$$

$${a}^{*} = 20.82$$

$${b}^{*} = 16.62$$

Paint card over black (measured using a Byk-mac I spectrophotometer, measurement angle) 15°:

$${L}^{*} = 72.03$$

$${a}^{*} = 74.63$$

$${b}^{*} = 21.35$$

Paint card over white (measured using a Byk-mac I spectrophotometer measurement angle) 45°:

$${L}^{*} = 27.58$$

$${a}^{*} = 34.52$$

$${b}^{*} = 29.50$$

The pigment according to the invention has the following PSD (particle size distribution) values (measured using a Malvern Mastersizer 3000):

$${D}_{10} = 9.4 μm$$

$${D}_{50} = 19.5 μm$$

$${D}_{90} = 35. μm$$

### Comparative Example 1: Example 1 from EP 1 681 318 A2

100 g of silica flakes (average flake thickness: 365 nm) are heated to 75° C. in 2 l of deionised water. 1120 ml of FeCl₃ solution (corresponds to 130% of Fe₂O₃) are added with stirring. The pH of the reaction mixture is kept constant at 3 by addition of sodium hydroxide solution (30%). After addition of the FeCl₃ solution, the pH is increased to 5 using sodium hydroxide solution (30%). The product is filtered off and rinsed with deionised water. After drying at 110° C., calcination is carried out at 800° C.

Paint card over black (measured using a Byk-mac I spectrophotometer, measurement angle) 75°:

$${L}^{*} = 14.62$$

$${a}^{*} = 21.85$$

$${b}^{*} = 18.67$$

Paint card over black (measured using a Byk-mac I spectrophotometer, measurement angle) 15°:

$${L}^{*} = 74.97$$

$${a}^{*} = 74.57$$

$${b}^{*} = 41.77$$

Paint card over white (measured using a Byk-mac I spectrophotometer, measurement angle) 45°:

$${L}^{*} = 28.69$$

$${a}^{*} = 35.67$$

$${b}^{*} = 34.18$$

The pigment according to Comparative Example 1 has the following PSD (particle size distribution) values (measured using a Malvern Mastersizer 3000):

$${D}_{10} = 8.9 μm$$

$${D}_{50} = 18.9 μm$$

$${D}_{90} = 34.9 μm$$

The pigment from Example 1 is particularly suitable for cosmetic applications and food applications owing to its colour purity and luminance.

In a direct comparison of the b* value (measured using a Byk-mac I spectrophotometer)—as a measure of the blue content (the lower the b* value, the higher the blue content)—it is clear at the 3 typical viewing angles for pearlescent pigments that the pigment according to Example 1 is significantly bluer than the pigment from Comparative Example 1 at all 3 viewing angles:

| | Paint card | Paint card | Paint card |
|---|---|---|---|
| b* | over black | over black | over white |
| value | 75° | 15° | 45° |
| Example 1 | 16.62 | 21.35 | 29.50 |
| Comparative | 18.67 | 41.77 | 34.18 |
| Example 1 | | | |

### Example 2—Post-Coating of Pigment According to Example 1

A 2% by weight aqueous solution comprising 2.55 g of sodium hypophosphate (NaH₂PO₂*H₂O) is added at room temperature with stirring to a 5% by weight aqueous solution comprising 3.90 g of zirconium oxychloride (ZrOCl₂*8H₂O) sufficiently slowly that no white precipitate forms. 4.1 g of 35% by weight hydrochloric acid are added to the clear solution obtained in this way, producing a mixed solution of zirconium oxychloride and sodium hypophosphate. The pigment according to Example 1 is suspended in 1000 ml of deionised water. 2.95 g of cerium (III) chloride (CeCl₃*7H₂O) are added to the suspension, which is kept at about 70° C. and adjusted to pH 2.5 using 10% HCl. The solution of zirconium oxychloride and sodium hypophosphate is added over 60 minutes, during which the pH is kept constant at 2.5 using 10% NaOH. The pH is subsequently increased to 7.0 over the course of 60 minutes by addition of 10% NaOH. 1.5 g of 3-aminopropyltrimethoxysilane (CAS No. 13822-56-5), 1.5 g of 3-glycidyloxypropyltrimethoxysilane (CAS No. 2530-83-8) and 0.5 g of n-hexyltrimethoxysilane (CAS No. 3069-19-0) are subsequently added to the suspension over the course of 15 minutes in each case, during which the pH is kept constant at 7.0 using 10% HCl or 10% NaOH. The pigment surface-treated in this way is filtered off, washed with water, dried at 140° C. and sieved (325 mesh).

The effect pigment has very good water and weather resistance.

### USE EXAMPLES

### Example A1: Lipstick

| | | | |
|---|---|---|---|
| Raw | Source of | INCI | |
| material | supply | EU | [%] |

Phase A

| | | | |
|---|---|---|---|
| Interference | (1) | CI 77491, SILICA | 11.60 |
| pigment according | | | |
| to Example 1 | | | |
| RonaFlair ® White | (1) | SYNTHETIC SAPPHIRE | 10.00 |
| Sapphire | | | |
| Covapate Unired | (2) | RICINUS COMMNUNIS | 1.00 |
| NA 3781 | | SEED OIL, CI 77491 | |

Phase B

| | | | |
|---|---|---|---|
| Oxynex ®K Liquid | (1) | PEG-8, TOCOPHEROL, | 0.05 |
| | | ASCORBYL PALMITATE | |
| | | ASCORBIC ACID, CITRIC | |
| | | ACID | |
| Mirasil ® Stearyl DM | (3) | STEARYL DIMETHICONE | 2.00 |
| Kaboguard ® | (4) | ACRYLATES/ETHYLHEXYL | 3.40 |
| 50AMP-G | | ACRYLATE COPOLYMER, | |
| | | AQUA, AMINOMETHYL | |
| | | PROPANOL | |
| Bentone Gel ISD V | (5) | ISODODECANE, | 3.50 |
| | | DISTEARDIMONIUM | |
| | | HECTORITE, PROPYLENE | |
| | | CARBONATE | |
| JBC WAX 200 | (6) | SYNTHETIC CANDELILLA | 6.50 |
| | | WAX | |
| Lanol 99 | (7) | ISONONYL | 9.40 |
| | | ISONONANOATE | |
| MAKIGREEN ™ | (8) | ORYZA SATIVA, ORYZA | 14.00 |
| VELVET WAX | | SATIVA BRAN OIL, | |
| | | HELIANTHUS ANNUUS, | |
| | | RHUS SUCCEDANEA | |
| | | FRUIT WAX | |
| Cetiol ® Ultimate | (9) | UNDECANE, TRIDECANE | 18.00 |
| PURESIL ™ TMS | (3) | DIMETHICONE/TRI- | add 100 |
| DM 30 | | METHYLSILOXY SILICATE | |

Phase C

| | | |
|---|---|---|
| FRAGRANCE | PARFUM | q.s. |

The constituents of phase B are heated to 80° C. and melted. The raw materials of Phase A are added and everything is stirred thoroughly. The lipstick composition is then stirred in the casting apparatus, which is held at a temperature of 75° C., until it no longer contains any air bubbles. The homogeneous melt is poured into the casting mould, which has been preheated to 55° C. The moulds are then cooled (about 1 hour), the castings are removed cold and placed in the lipstick cases. The lipsticks are then briefly flame-treated.

### Manufacturer

(1) Merck KGaA/EMD Performance Materials Corp.
(2) Sensient Cosmetic Technologies
(3) Elkem
(4) Kobo Products
(5) Elementis Specialties
(7) Seppic (6) Alpha Quimica
(8) PIC Quimica
(9) BASF AG

### Example A2: Facial Toner (Micellar Water)

| | | | |
|---|---|---|---|
| Raw | Source of | INCI | |
| material | supply | EU | [%] |

Phase A

| | | | |
|---|---|---|---|
| Glycerol 85% | (1) | GLYCERIN, AQUA | 5.00 |
| Solagum ™ AX | (2) | ACACIA SENEGAL GUM, | 0.80 |
| | | XANTHAN GUM | |
| Demineralised | | AQUA | add 100.00 |
| water | | | |

Phase B

| | | | |
|---|---|---|---|
| Schercoteric ™ C-2 | (3) | DISODIUM | 1.00 |
| 50LT surfactant | | COCOAMPHODIACETATE | |
| Pluracare ® L 64 G | (4) | STEARYL DIMETHICONE | 0.20 |

Phase C

| | | | |
|---|---|---|---|
| RonaCare | (1) | AQUA, POLYSIPHONIA | 1.00 |
| RenouMer Cos | | ELONGATA EXTRACT, | |
| | | SODIUM BENZOATE, | |
| | | CITRIC ACID | |

Phase D

| | | | |
|---|---|---|---|
| RonaFlair ® White | (1) | SYNTHETIC SAPPHIRE | 3.00 |
| Sapphire | | | |
| Interference | (1) | CI 77491, Silica | 0.50 |
| pigment according | | | |
| to Example 1 | | | |
| Sodium hydroxide, | (1) | AQUA, SODIUM | 0.20 |
| 10% | | HYDROXIDE | |

Phase E

| | | | |
|---|---|---|---|
| Lipocol ® HCO-40 | (5) | PARFUM | 0.05 |
| FRAGRANCE | (1) | PEG-40 HYDROGENATED | q.s. |
| | | CASTOR OIL | |
| Preservatives | (1) | PRESERVATIVES | q.s. |

### Preparation

Phase B is added to Phase A with vigorous stirring. After homogenisation, phase C is added and the pH is adjusted to 5.0-5.5. Phase D is slowly added to the premixed phase E and everything is mixed with phases A/B/C with stirring.

### Manufacturer

(1) Merck KGaA/EMD Performance Materials Corp.
(2) Seppic
(3) Lubrizol
(4) BASF AG
(5) Vantage Personal Care

### Example A3: Eye Shadow

| | | | |
|---|---|---|---|
| Raw | Source of | INCI | |
| material | supply | EU | [%] |

Phase A

| | | | |
|---|---|---|---|
| Ronastar ® Blue | (1) | SYNTHETIC SAPPHIRE, | 37.00 |
| Lights | | CI 77891, SILICA, TIN | |
| | | OXIDE | |
| Interference | (1) | | 12.00 |
| pigment according | | | |
| to Example 1 | | | |

Phase B

| | | | |
|---|---|---|---|
| RonaFlair ® Mica M | (1) | MICA | 20.60 |
| RonaFlair ® Soft | (1) | SYNTHETIC | 10.00 |
| Sphere | | FLUORPHLOGOPITE, | |
| | | SILICA | |
| Mg stearate | (2) | MAGNESIUM STEARATE | 2.00 |
| DOWSIL ™ 9701 | (3) | DIMETHICONE/VINYL | 1.40 |
| Cosmetic Powder | | DIMETHICONE | |
| | | CROSSPOLYMER, | |
| | | SILICA | |

Phase C

| | | | |
|---|---|---|---|
| Squalane | (2) | ISONONYL | add 100.00 |
| | | ISONONANOATE | |
| Eutanol G | (4) | ORYZA SATIVA, ORYZA | 4.25 |
| | | SATIVA BRAN OIL, | |
| | | HELIANTHUS ANNUUS, | |
| | | RHUS SUCCEDANEA | |
| | | FRUIT WAX | |
| KF-96A-6cs | (5) | UNDECANE, TRIDECANE | 14.25 |
| Preservative | | DIMETHICONE/TRI- | q.s. |
| | | METHYLSILOXY | |
| | | SILICATE | |

Mix the components of phase B and add phase A. Add the mixture of phase C with stirring. Press the powder at 40-50 bar.

### Manufacturer

(1) Merck KGaA/EMD Performance Materials Corp.
(2) China Chemical Reagent
(3) Dow Corning
(4) BASF AG
(5) Shin Etsu Silicones

### Example A4: Lip Gloss

| | | | |
|---|---|---|---|
| Raw | Source of | INCI | |
| material | supply | EU | [%] |

Phase A

| | | | |
|---|---|---|---|
| Interference | | | 1.00 |
| pigment | | | |
| Timiron ® Supersilk | (1) | MICA, CI 77491 | 2.50 |
| MP-1005 | | | |
| Colourona ® Fine | (1) | CI 77891, MICA, CI 77491 | 5.00 |
| Gold MP-20 | | | |
| Covanol White | (2) | CI 77891, | 0.50 |
| ON 9788 | | OCTYLDODECANOL, | |
| | | STEARIC ACID, ALUMINUM | |
| | | HYDROXIDE, | |
| | | POLYHYDROXYSTEARIC | |
| | | ACID | |

Phase B

| | | | |
|---|---|---|---|
| Eusolex ®2292 | (1) | MICA | 7.50 |
| Eusolex ® OS | (1) | SYNTHETIC | 5.00 |
| | | FLUORPHLOGOPITE, SILICA | |
| RonaCare ® | (1) | MAGNESIUM STEARATE | 0.40 |
| Bisabolol nat. | | | |
| Oxynex ® ST liquid | (1) | DIMETHYLHEXYL | 0.30 |
| | | SYRINGYLIDENEMALONATE, | |
| | | CAPRYLIC/CAPRIC | |
| | | TRIGLYCERIDE | |
| Oxynex ® K liquid | (1) | PEG-8, TOCOPHEROL, | 0.50 |
| | | ASCORBYL PALMITATE | |
| | | ASCORBIC ACID, CITRIC | |
| | | ACID | |
| Finsolv TPP | (3) | C12-15 ALKYL BENZOATE, | 3.00 |
| | | DIPROPYLENE GLYCOL | |
| | | DIBENZOATE | |
| Myritol 312 | | | 3.00 |
| Eutanol G | | | 4.00 |
| Cetiol CC | | | 4.60 |
| Miglyol ® Gel 840 B | | | 10.00 |
| Softisan 649 | | | add 100.00 |
| Preservative | | | q.s. |

Phase C

| | | | |
|---|---|---|---|
| Aerosil ® R 812 S | (8) | SILICA SILYLATE | 2.00 |

Mix the components of Phase B with stirring. Add the components of Phase A and then Phase C with stirring. Finally, transfer the mixture into a suitable container.

### Manufacturer

(1) Merck KGAA/EMD Performance Materials Corp.
(2) Sensient Cosmetic Technologies
(3) Innospec
(4) BASF AG
(5) Croda
(6) Sasol Germany GmbH
(7) IOI Oleo GmbH
(8) Evonik Nutrition & Care GmbH

### Example A5: Nail Varnish

| Raw | Source of | INCI | |
|---|---|---|---|
| material | supply | EU | [%] |
| Interference | | | 4.00 |
| pigment according | | | |
| to Example 1 | | | |
| Nail varnish base | (1) | ETHYL ACETATE, BUTYL | 96.00 |
| 12897 | | ACETATE, | |
| | | NITROCELLULOSE, | |
| | | PHTHALIC | |
| | | ANHYDRIDE/TRIMELLITIC | |
| | | ANHYDRIDE/GLYCOLS | |
| | | COPOLYMER, ACETYL | |
| | | TRIBUTYL CITRATE, | |
| | | ISOPROPYL ALCOHOL, | |
| | | STEARALKONIUM | |
| | | HECTORITE, ADIPIC | |
| | | ACID/NEOPENTYL | |
| | | GLYCOL/TRIMELLITIC | |
| | | ANHYDRIDE | |
| | | COPOLYMER | |

### Preparation

The interference pigment is mixed thoroughly with the varnish base (1000 rpm) for 10 minutes.

### Manufacturer

(1) International Lacquers SA

### Example A6: Lip Gloss

| | | | |
|---|---|---|---|
| Raw | Source of | INCI | |
| material | supply | EU | [%] |

Phase A

| | |
|---|---|
| Interference | 4.00 |
| pigment according | |
| to Example 1 | |

Phase B

| | | | |
|---|---|---|---|
| VPT162 - | (2) | RICINUS COMMUNIS | 40.00 |
| Vegetable | | SEED OIL, | |
| Petrolatum | | HYDROGENATED | |
| Transparent 162 | | CASTOR OIL, | |
| | | COPERNICIA CERIFERA | |
| | | CERA, CERA ALBA | |
| Kahlresin 5723 MB | (1) | GLYCERYL ROSINATE, | 30.00 |
| | | OCTYLDODECANOL | |
| Castor oil, pressed | (2) | RICINUS COMMUNIS | 15.50 |
| | | SEED OIL | |
| Refined apricot oil | (3) | PRUNUS ARMENIACA | 10.00 |
| | | KERNEL OIL | |

Phase C

| | | | |
|---|---|---|---|
| all-rac-alpha- | (1) | TOCOPHEROL | 0.50 |
| Tocopherol | | | |

### Preparation

Heat phase B to 70° C. and homogenise with stirring. Allow to cool slowly to 50° C. with stirring and add the interference pigment (phase A). Allow to cool further to 35° C. and add phase C and then transfer into suitable containers.

### Manufacturer

(1) Merck KGaA/EMD Performance Materials Corp.
(2) DKSH GmbH
(3) Kahl GmbH & Co. KG
(4) IES Lab

### Example A7: Rouge

| | | | |
|---|---|---|---|
| Raw | Source of | INCI | |
| material | supply | EU | [%] |

Phase A

| | | | |
|---|---|---|---|
| Interference | | | 7.00 |
| pigment according | | | |
| to Example 1 | | | |
| Timiron ® Glam | (1) | Alumina, CI 77891, TIN | 3.00 |
| Silver | | OXIDE | |
| RonaFlair ® Satin | (1) | ILLITE | 20.00 |

Phase B

| | | | |
|---|---|---|---|
| Oxynex ® L-CV | (1) | ALCOHOL, | 0.30 |
| Liquid | | TOCOPHEROL, | |
| | | CAPRYLIC/CAPRIC | |
| | | TRIGLYCERIDE, | |
| | | ASCORBYL PALMITATE, | |
| | | ASCORBIC ACID, CITRIC | |
| | | ACID | |
| Organic jojoba oil | (2) | SIMMONDSIA | 2.00 |
| | | CHINENSIS SEED OIL | |
| Crodamol ™ GTCC | (3) | CAPRYLIC/CAPRIC | 7.00 |
| MBAL-LQ-(MV) | | TRIGLYCERIDE | |
| Eutanol G | (4) | OCTYLDODECANOL | 12.00 |
| Cetiol OE | (4) | DICAPRYLYL ETHER | 16.00 |

Phase C

| | | | |
|---|---|---|---|
| Bentone gel | (5) | PROPYLENE | 32.70 |
| | | CARBONATE, C12-15 | |
| | | ALKYL BENZOATE, | |
| | | STEARALKONIUM | |
| | | HECTORITE | |

### Preparation

Mix the constituents of phase A and phase C separately. Add phase C to phase B and mix. Add phase A to the mixture of phases B and C and homogenise everything using the Ultra-Turrax at 8000 rpm.

### Manufacturer

(1) Merck KGaA/EMD Performance Materials Corp.
(2) IES Lab
(3) Croda
(4) BASF AG
(5) Nordmann, Rassmann GmbH

### Example A8: Lipstick

| | | | |
|---|---|---|---|
| Raw | Source of | INCI | |
| material | supply | EU | [%] |

Phase A

| | | | |
|---|---|---|---|
| Interference | | | 18.00 |
| pigment according | | | |
| to Example 1 | | | |
| RonaFlair ® LDP | (1) | SODIUM POTASSIUM | 5.00 |
| White | | ALUMINUM SILICATE, CI | |
| | | 77891, SILICA | |

Phase B

| | | | |
|---|---|---|---|
| RonaCare ® | (1) | BISABOLOL | 0.50 |
| bisabolol nat. | | | |
| RonaCare ® Poppy | (1) | CAPRYLIC/CAPRIC | 0.50 |
| SE | | TRIGLCERIDE, PAPAVER | |
| | | RHOEAS EXTRACT, | |
| | | TOCOPHEROL | |
| Oxynex ® L-CV | (1) | ALCOHOL, | 0.50 |
| Liquid | | TOCOPHEROL, | |
| | | CAPRYLIC/CAPRIC | |
| | | TRIGLYCERIDE, | |
| | | ASCORBYL PALMITATE, | |
| | | ASCORBIC ACID, CITRIC | |
| | | ACID | |
| Cetiol SB 45 | (2) | BUTYROSPERMUM | 3.00 |
| | | PARKII BUTTER | |
| Orange peel wax | (3) | CITRUS AURANTIUM | 3.00 |
| | | DULCIS (ORANGE) PEEL | |
| | | WAX | |
| Sunflower wax | (3) | HELIANTHUS ANNUUS | 5.00 |
| | | SEED CERA | |
| Beeswax white, | (4) | CERA ALBA | 12.00 |
| organic | | | |
| Myritol ®318 | (2) | CAPRYLIC/CAPRIC | |
| | | TRIGLCERIDE | |
| Organic jojoba oil | (5) | SIMMONDSIA | 18.00 |
| | | CHINENSIS SEED OIL | |
| Castor oil, pressed, | (4) | RICINUS COMMUNIS | 20.00 |
| organic | | SEED OIL | |

### Preparation

The constituents of phase B are heated to 80-85° C. and melted. The raw materials of phase A are added, and everything is mixed thoroughly. The lipstick composition is then stirred in the casting apparatus, which is held at a temperature of 80° C., until it no longer contains any air bubbles. The homogeneous melt is poured into the casting mould, which has been preheated to 55° C. The moulds are then cooled (about 1 hour), the castings are removed cold and placed in the lipstick cases. Finally, the lipsticks are briefly flame-treated.

### Manufacturer

(1) Merck KGAA/EMD Performance Materials Corp.
(2) BASF AG
(3) Koster Keunen Holland BV
(4) Henry Lamotte Oils GmbH
(5) IES Lab

### Example A9: Lip Gloss

| | | | |
|---|---|---|---|
| Raw | Source of | INCI | |
| material | supply | EU | [%] |

Phase A

| | | | |
|---|---|---|---|
| Interference pigment | | | 1.00 |
| according to | | | |
| Example 1 | | | |
| RonaCare ® Poppy | (1) | CAPRYLIC/CAPRIC | 0.50 |
| SE | | TRIGLCERIDE, | |
| | | PAPAVER RHOEAS | |
| | | EXTRACT, | |
| | | TOCOPHEROL | |
| RonaCare ® | (1) | BISABOLOL | 0.10 |
| bisabolol nat. | | | |

Phase B

| | | | |
|---|---|---|---|
| Oxynex ® ST Liquid | (1) | DIETHYLHEXYL | 0.10 |
| | | SYRINGYLIDENEMAL | |
| | | ONATE, | |
| | | CAPRYLIC/CAPRIC | |
| | | TRIGLYCERIDE | |
| El shea butter | (2) | SHEA BUTTER | 3.00 |
| LexFeel Shine | (3) | PROPYLENE | 4.00 |
| | | GLYCOL | |
| | | DIBENZOATE | |
| Beeswax | (4) | CERA ALBA | 4.00 |
| Myritol 318 | (5) | CAPRYLIC/CAPRYLIC | 5.40 |
| | | TRIGLYCERIDE | |
| Crystal Crown ® | (2) | RICINUS COMMUNIS | 8.90 |
| castor oil | | (CASTOR) SEED OIL | |
| Cetiol ® 868 | (5) | ETHYLHEXYL | 10.00 |
| | | STEARATE | |
| Versagel ® ME 750 | (6) | HYDROGENATED, | 24.00 |
| | | POLYISOBUTENE, | |
| | | ETHYLENE/PROPYLENE/ | |
| | | STYRENE COPLYMER, | |
| | | BUTYLENE/ETHYLENE/ | |
| | | STYRENE COPOLYMER; | |
| | | BHT | |
| Indopol H-100 | (7) | POLYBUTENE | 30.00 |

### Preparation

Heat phase B to 90-95° C. with stirring until a clear emulsion forms. Cool the temperature down to 55-60° C. and add phase A with stirring. Cool further to 45-50° C. and then transfer into suitable containers.

### Manufacturer

(1) Merck KGaA/EMD Performance Materials Corp.
(2) Essential Ingredients
(3) INOLEX Chemical Company
(4) Strahl & Pitsch
(5) BASF AG
(6) Penreco
(7) Ineos Oligomers

### Example A10: Production of Hard Caramels

| Raw material | % | Sources of supply: |
|---|---|---|
| Sugar | 41% | Südzucker |
| Water | 17.118% | |
| Glucose syrup | 41% C* Sweet | Cerestar, Krefeld |
| Interference pigment | 0.082% (0.1% based | Merck KGaA, Darmstadt |
| according to | on the casting | |
| Example 1 | composition) | |
| E 104 1:100 dil. | 0.4% Sikovit | BASF, Ludwigshafen |
| Aroma | 0.4% | Dragaco, Holzminden |
| | (banana 9/030388) | |

The sugar is heated to 100° C. with the water, and glucose syrup is then added. The solution is then heated to 145° C. After addition of the interference pigment, the colouring solution and the flavouring, the caramel solution is poured into greased moulds using a funnel. Finally, it is allowed to cool for two hours. The interference pigment can either be mixed with the sugar or added as a mixture with the glucose syrup. This variant does not contain any acid, as this would cause excessive caramelisation.

### Example A11: Production of Gelatine Products

| Raw material | % | Sources of supply: |
|---|---|---|
| Water | 10.48% | |
| Sugar | 31.45% | Südzucker |
| Glucose syrup | 31.45% C*Sweet | Cerestar, Krefeld |
| Interference pigment | 0.38% (0.4% based | Merck KGaA, |
| according to Example | on the casting | Darmstadt |
| 1 | composition) | |
| Citric acid 1:1 diluted | 2.51% | Merck KGaA, |
| | | Darmstadt |
| Gelatine | 7.86% 260 Bloom | DGF, Eberbach |
| Water | 15.748% | |
| Aroma | 0.122% (blackcurrant | Dragoco, Holzminden |
| | 9/695750) | |

Firstly, the gelatine is softened using twice the amount of water at 60° C. The sugar and water are heated to 100° C., then the glucose syrup is added. The mixture is heated further to 120° C. and then allowed to cool to about 85° C. The interference pigment, the citric acid, the flavouring and the gelatine solution are stirred in and the deaerated gelatine mixture is transferred into greased moulds using a funnel. The product is allowed to cool for about 16 hours.

### FURTHER EMBODIMENTS

The bluish-red interference pigment can again be mixed directly with the sugar or introduced with the glucose syrup.
Instead of pouring into moulds, it is also possible to use the traditional technique using negative moulds in moulding powder for the production of gelatine articles.

### Example A12: Coating of Tablets

a) Initial weight 1 kg of white tablets d=8 mm, W=200 mg

Solution for film coating:

| | | |
|---|---|---|
| 6% | Sepifilm Lp10 | Seppic |
| | (mixture of hydroxypropylmethyl- | |
| | cellulose, stearic acid and micro- | |
| | crystalline cellulose) | |
| 5% | Interference pigment according | Merck KGaA, Darmstadt |
| | to Example 1 | |
| 89% | Water | |

Total amount applied: 200 g

This corresponds to 1.2 mg of polymer/cm² of tablet surface

### Preparation of the Film Coating Solution:

The interference pigment is stirred into water. Additional dyes are then optionally added. Finally, the film former (HPMC) is sprinkled into the suspension. Due to the increasing viscosity, the stirring speed must also be increased correspondingly. After about 40-60 minutes, the HPMC has dissolved completely and the solution can then be sprayed onto the tablets.
The spray application is carried out by means of a standard coating method.

### Example A13: Ceramics

### 1) Preparation of the Printing Paste:

For the preparation of fine colour screens and relief-like prints on ceramic substrates by means of ceramic colours, screen-printing oils are employed which prevent the colour pastes from running after printing and give rise to prints with sharp contours. To this end, use is made of additives to the known binders which consist of finely divided natural or synthetic waxes and/or finely divided inorganic silicate or oxidic substances which can be incorporated into the silicate framework of the flux during firing. The interference pigment according to Example 1 is weighed out and homogenised with the corresponding amount of frit and the printing medium (in the examples, 221-ME screen printing oil and Screenprint Bulk 803035 MR—both commercially available products from Ferro—are employed) for a series of experiments (see Table 1).

In Examples 1 to 19 below, a frit having the following composition

| Frit | CaO | Na₂O | K₂O | BaO | Al₂O₃ | SiO₂ | B₂O₃ |
|---|---|---|---|---|---|---|---|
| % by | 9.7 | 5.2 | 1.1 | 1.3 | 10.1 | 69.6 | 3.0 |
| weight | | | | | | | |

is weighed out and homogenised.

The initial weight of the corresponding raw materials for the preparation of the printing paste, i.e. interference pigment, frit and printing oil for paste preparation, is shown in the table below:

TABLE 1

| | Interference | | | | | |
|---|---|---|---|---|---|---|
| Example | pigment | | Printing | Pasting | W_{pigm} in | W_{pigm} in |
| No. | (Example 1) | Frit | oil | ratio | the solid | the paste |
| 1 | 1.5 g | 3.5 g | 7.0 g | 10:14 | 30.00 | 12.50 |
| 2 | 1.0 g | 3.5 g | 7.0 g | 10:15.6 | 22.22 | 8.70 |
| 3 | 0.8 g | 3.5 g | 7.0 g | 10:16.3 | 18.60 | 7.08 |
| 4 | 0.6 g | 3.5 g | 7.0 g | 10:17.1 | 14.63 | 5.41 |
| 5 | 1.5 g | 3.5 g | 9.0 g | 10:18 | 30.00 | 10.71 |
| 6 | 1.0 g | 3.5 g | 9.0 g | 10:20 | 22.22 | 7.41 |
| 7 | 0.8 g | 3.5 g | 9.0 g | 10:20.9 | 18.60 | 6.02 |
| 8 | 0.6 g | 3.5 g | 9.0 g | 10:22 | 14.63 | 4.58 |
| 9 | 1.5 g | 2.0 g | 7.0 g | 10:20 | 42.86 | 14.29 |
| 10 | 1.5 g | 1.5 g | 7.0 g | 10:23.3 | 50.00 | 15.00 |
| 11 | 1.5 g | 1.0 g | 7.0 g | 10:28 | 60.00 | 15.79 |
| 12 | 1.5 g | 0.5 g | 7.0 g | 10:35 | 75.00 | 16.67 |
| 13 | 5.0 g | 0.0 g | 7.0 g | 10:14 | 100.00 | 41.67 |
| 14 | 2.0 g | 3.0 g | 7.0 g | 10:14 | 40.00 | 16.67 |
| 15 | 3.8 g | 1.2 g | 7.0 g | 10:14 | 76.00 | 31.67 |
| 16 | 4.3 g | 0.7 g | 7.0 g | 10:14 | 86.00 | 35.83 |
| 17 | 4.5 g | 0.5 g | 7.0 g | 10:14 | 90.00 | 37.50 |
| 18 | 4.5 g | 0.2 g | 7.0 g | 10:14 | 96.00 | 40.00 |
| 19 | 4.8 g | 0.1 g | 7.0 g | 10:14 | 98.00 | 40.83 |

The following steps 2-4 are independent of the composition of the printing paste.

### 2) Printing of the Tiles

The printing paste obtained can be applied to tiles using standard printing methods, slip methods, spray application or transfer printing. In all cases, the printed tile is dried in a drying cabinet or fume hood at temperatures of 60-110° C. in order to evaporate the solvent present in the printing oil. In the examples according to the invention, the printing paste is applied to the tiles by means of a squeegee and printing screen.

### 3) Firing of the Printed Tiles

The printed and dried tile is then fired in the firing oven by means of a temperature profile as shown in FIG. 6.
180 min: heating to 1100° C.,
3 min: hold at 1100° C.,
120 min: rapid cooling to 600° C.,
300 min: slow cooling to room temperature.

The glazed tiles of Examples 1 to 19 are distinguished by the fact that the desired optical effects are stable and accessible reproducibly when used at high temperatures >1100° C.

1. An interference pigment, comprising SiO₂ flakes coated with a crystallite of alpha-Fe₂O₃.

2. The interference pigment of claim 1, wherein the SiO₂ flakes have a diameter of 1-250 μm.

3. The interference pigment of claim 1, wherein the SiO₂ flakes have a thickness of 250-500 nm.

4. The interference pigment of claim 1, wherein the crystallite of alpha-Fe₂O₃ has an average size of ≥45 nm.

5. The interference pigment of claim 1, having an average total thickness of 494 nm +/−109 nm.

6. The interference pigment of claim 1, further comprising an organic or inorganic post-coating.

7. A process for the preparation of an interference pigment that comprises SiO₂ flakes coated with a crystallite of alpha-Fe₂O₃, comprising coating the SiO₂ flakes with the crystallites of alpha-Fe₂O₃ by a wet-chemical process in a fluidised bed, by a CVD process, by a PVD process, or by any combination of said processes.

8. A paint, coating, ink, paper, plastic, film, cosmetic formulation, button paste, pigment mixture, ceramic material, ceramic colour, glaze, engobe, enamel, glass, absorber for the laser marking of plastics, food color, food finish, document security feature, seed coloring, RADAR application or solar cell coloring comprising the interference pigment of claim 1.

9. A formulation comprising the interference pigment of claim 1 and at least one further constituent.

10. The formulation of claim 9, wherein the at least one further constituent comprises one or more constituents selected from the group consisting of absorbents, astringents, antimicrobial substances, antioxidants, antiperspirants, antifoams, antidandruff active compounds, antistatics, flavourings, binders, biological additives, bleaching agents, chelating agents, deodorants, printing oils, emollients, emulsifiers, emulsion stabilisers, dyes, humectants, film formers, frits, fillers, fragrances, flavours, insect repellents, preservatives, corrosion inhibitors, cosmetic oils, solvents, oxidants, perfumes, plant constituents, proteins, buffer substances, reducing agents, abrasives, sweeteners, surfactants, propellant gases, opacifiers, UV filters, UV absorbers, denaturing agents, aloe vera, avocado oil, coenzyme Q10, green tea extract, organic pigments, inorganic pigments, viscosity regulators, vitamins, enzymes, trace elements, and carbohydrates.
